(19) United States
(12) Patent Application Publication
(10) Pub. No.: US 2016/0045266 A1
(43) Pub. Date: Feb. 18, 2016

(54) STETHOSCOPE COVER

(71) Applicants: Robert DEPORTO, Brooklyn, NY (US); Basit Qayyum, New York, NY (US)

(72) Inventors: Robert DEPORTO, Brooklyn, NY (US); Basit Qayyum, New York, NY (US)

(21) Appl. No.: 14/825,933

(22) Filed: Aug. 13, 2015

Related U.S. Application Data
(60) Provisional application No. 62/037,459, filed on Aug. 14, 2014.

(51) Int. Cl. A61B 19/02; B65B 7/02; B65B 51/02; B65D 83/08

(52) U.S. Cl. CPC A61B 19/026; B65D 83/0894; B65B 7/02; B65B 51/02

(57) ABSTRACT

Aspects relate to methods and systems that provide a protective cover for medical devices, such as a stethoscope, to prevent the transmission of infections and/or bacteria from one individual to another. In some examples, the protective cover may be an environmentally friendly, disposable barrier, including but not limited to a bacteriostatic material. In some examples, the protective cover may be for single use, such that the protective cover may be disposed of after use. In an aspect, the protective cover may have a tab at the bottom, near the diaphragm, for easy removal. Thus, the protective cover disclosed in aspects of the present application allows full capability of the stethoscope to transmit quality sound, while preventing the transmission of infections and/or bacteria from one individual to another.

### CROSS-REFERENCE TO RELATED APPLICATION(S)

This application claims priority to U.S. Provisional Application Ser. No. 62/037,459, entitled, “STETHOSCOPE COVER,” and filed on Aug. 14, 2014, which is assigned to the assignee hereof and hereby expressly incorporated by reference herein in its entirety.

### BACKGROUND

Medical equipment used on multiple patients without proper aseptic techniques or cleaning can pose a risk of spreading contact pathogens and transmission of infections and/or bacteria from one individual to another.

Although methods for preventing transmission of infections and/or bacteria from one individual to another via stethoscope may be available, none of the conventional methods provide a system that is effective or easy to use. For example, simply covering only the stethoscope head or tubing shaft may not prevent cross contamination from the examiner's gloved hand touching the auricular shaft and thus contaminating the stethoscope. Accordingly, not only are other patients put at an elevated risk, but the practitioner himself/herself is also at an elevated risk because most practitioners either drape the stethoscope over their neck or fold the stethoscope into a pocket. Another alternative may include using a disposable stethoscope. However, disposable stethoscopes typically provide poor sound quality, and may be costly, and environmentally unfriendly. Another approach may include cleaning the stethoscope with an antibacterial agent in an attempt to prevent disease transmission. This approach also may not be effective, however, as many stethoscopes contain grooves and ridges that will hinder the efficacy of sterilization through wiping.

Thus, there is a need in the art for preventing transmission of infections and/or bacteria through the use of a stethoscope on multiple patients.

### SUMMARY

Aspects relate to methods and systems that, among other things, provide a protective cover for medical devices, such as a stethoscope, to prevent the transmission of infections and/or bacteria from one individual to another. In some examples, the protective cover may be an environmentally friendly, disposable barrier, including but not limited to a bacteriostatic material. In an aspect, the protective cover may be for single use, such that the protective cover may be disposed of after use. In one or more examples, the protective cover may be made of non-biodegradable and/or biodegradable materials that cover the full length of the stethoscope, e.g., from diaphragm up to and including the auricular tubes, with a simple, releasable locking adhesive device. In an aspect, the protective cover may have a tab at the bottom, near the diaphragm, for easy removal. Thus, the protective cover allows full capability of the stethoscope to transmit quality sound, while preventing the transmission of infections and/or bacteria from one individual to another.

Additional advantages and novel features relating to aspects of the present invention will be set forth in part in the description that follows, and in part will become more apparent to those skilled in the art upon examination of the following or upon learning by practice thereof.

### DESCRIPTION OF THE FIGURES

In the drawings:

FIGS. 1A-1C illustrate an example protective cover for a stethoscope in accordance with an aspect;

FIG. 2 illustrates an example lateral view of a protective cover dispenser in accordance with an aspect;

FIG. 3 illustrates an example top view of a protective cover dispenser in accordance with an aspect; and

FIG. 4 illustrates an example top view of a protective cover dispenser in accordance with an aspect.

### DETAILED DESCRIPTION

Aspects relate to methods and systems that, among other things, provide a protective cover for a medical device, such as a stethoscope, to prevent the transmission of infections and/or bacteria from one individual to another. The protective cover may be an environmentally friendly, disposable barrier, such as but not limited to a bacteriostatic material. The protective cover may be for single use and then disposed of after use. The protective may be made of non-biodegradable and/or biodegradable materials that cover the full length of the stethoscope, e.g., from diaphragm up to and including the auricular tubes, with a simple, releasable locking adhesive device. In an aspect, the protective cover may have a tab at the bottom, near the diaphragm, for easy removal. Thus, the protective cover allows full capability of the stethoscope to transmit quality sound while preventing the transmission of infections and/or bacteria from one individual to another.

In an aspect, the protective cover may be a barrier placed on the stethoscope through a protective cover dispenser that holds one or more of the barriers. In some examples, the protective cover dispenser may be a plastic box that holds the barriers. The protective cover dispenser may include barriers inside the box in sets of 10 to 100 or more, positioned in a longitudinal fashion and removably attached to each other, so that a barrier may separate from the set when pulled out. For example, the barriers may be attached to one another with a perforation barrier between each barrier so that the barriers may separate from one another when pulled out. The stethoscope may be placed using an open inlet on top of the box and may slide through the exit in front or side and pulled out. The barrier may enclose the entire stethoscope, e.g., from diaphragm up to and including the auricular tubes, except for a region just proximal to and including the earpiece.

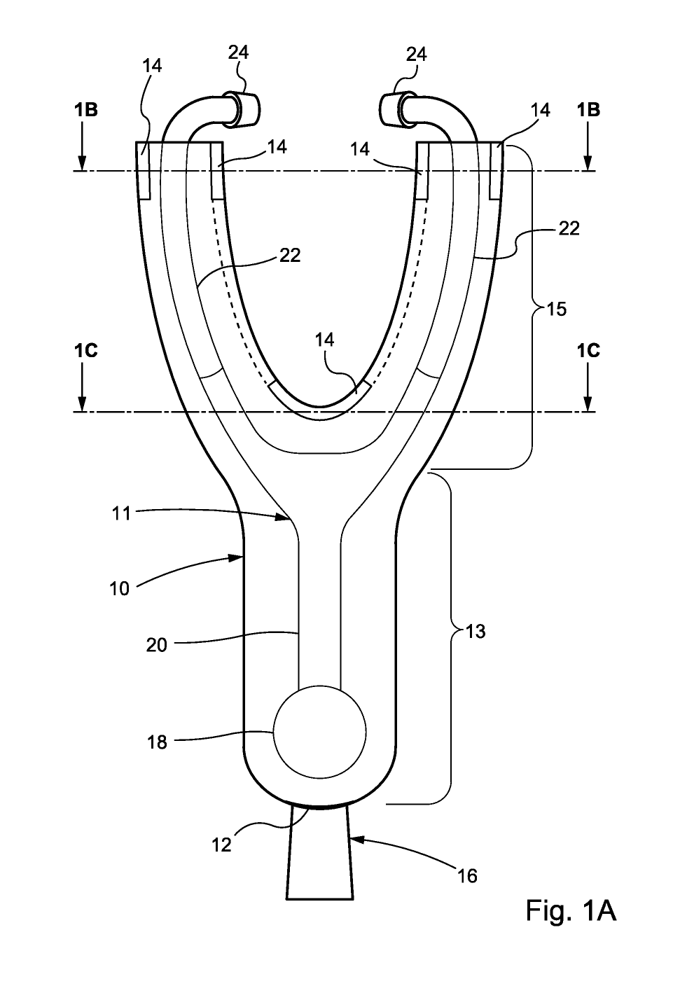

Referring now to FIG. 1A, illustrated therein is an example protective cover in accordance with an aspect. The protective cover 10 may be made of an impermeable material, which when applied encases the entire stethoscope 11 including the bell 18 and tubing 20 as well as the auricular tubes 22. In an aspect, protective cover 10 may include a distal portion 13 having a different configuration as compared to a proximal portion 15. Distal portion 13 may be configured to encase the bell 18 and, optionally, tubing 20 extending from bell 18, of the inserted stethoscope 11. In an aspect, distal portion 13 may include a seal 12 and may have a tubular shape. The distal portion 13 may be referred to as a distal first tubular portion of protective cover 10. Further, for example, proximal portion 15 of protective cover 10 may cover auricular tubes 22 extending from tubing 20. The proximal portion 15 may be referred to as a proximal second portion of protective cover 10. In some aspects, proximal portion 15 may cover some part of tubing 20. In one or more examples, the proximal portion 15 may be an extension of the same impermeable material as distal portion 13. Additionally or alternatively, the proximal portion 15, in some examples, may not be tubular but instead may be flat so as to allow auricular tubes 22 to be received within proximal portion 15, such as when stethoscope 11 is inserted within protective cover 10 from above. In an aspect, proximal portion 15 may contain one or more adhesive components 14 along the edges and at the ends (e.g., near the ear piece 24) which will allow the practitioner to pinch proximal portion 15 of protective cover 10 closed over the auricular tubes 22, resulting in an enveloping tube around auricular tubes 22. Additionally or alternatively, the adhesive 14 may extend the length of the inner portion of the protective cover 10 as illustrated by dashed line such that the seam of the protective cover 10 may be sealed, and thereby allowing the stethoscope 11 to be protected from being exposed to the elements. Thus, in some examples, once the stethoscope 11 has been inserted into the protective cover 10, a practitioner may pinch adhesives 14 on either side of the protective cover 10 to seal the cover around the stethoscope 11. In an aspect, proximal portion 15 may be folded around auricular tubes 22, thereby in combination with distal portion 13 encloses/protects the entire stethoscope 11, except for in a region proximal to earpieces 24. Further, in an aspect, protective cover 10 may include a tab 16 at the bottom end to allow easy grabbing and easy removal of protective cover 10 from stethoscope 11. Moreover, in an aspect, protective cover 10 may include an antibacterial and/or antifungal powder, such as on an outer surface of the materials, and may be dispensed in cartridges of, for example, 10 to 100 or more protective covers.

FIG. 1B illustrates a cross-sectional top-down view of the protective cover 10 applied to the stethoscope 11. As discussed above, the protective cover 10 may include one or more adhesive components 14 along the ends (e.g., near the ear piece 24) of the protective cover 10 which may allow the practitioner to pinch the adhesives 14 around the auricular tube 22 to secure the protective cover 10 around the stethoscope 11. Additionally or alternatively, as illustrated in FIG. 1C, the protective cover 10 may include one or more adhesives 14 at the open end of the proximal portion 15 to allow the open end of the protective cover 10 to be sealed at the seam. Thus, in accordance with various aspects of the present disclosure, a stethoscope 11 may be dropped into the open top of the protective cover 10, and thereafter a practitioner may be able to pinch one or more points around the proximal portion 15, for example, to seal the protective cover 10 around the stethoscope 11.

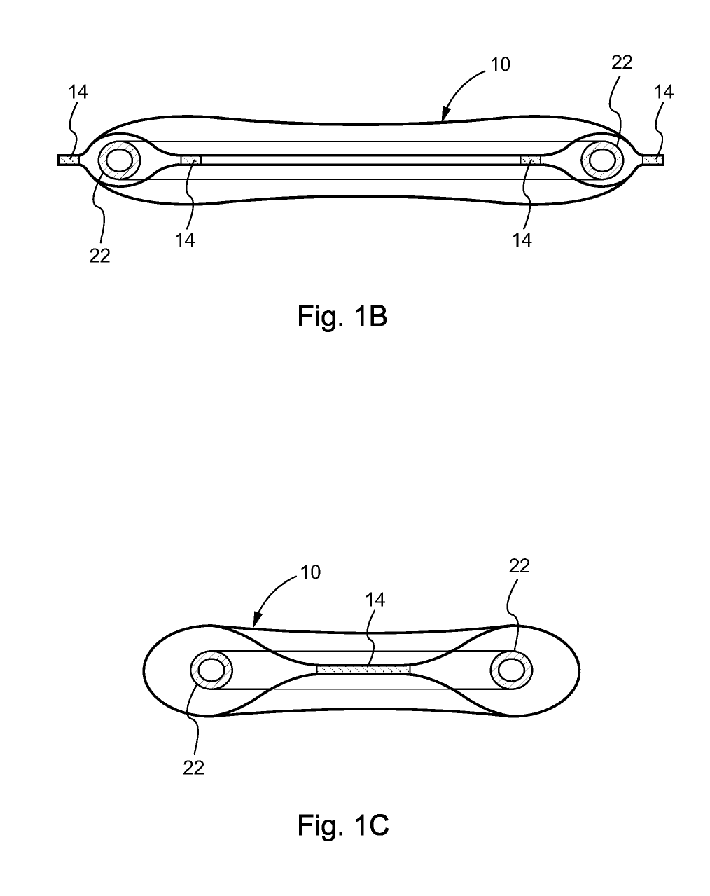

Referring now to FIG. 2, illustrated therein is a lateral view of a protective cover dispenser 26 in accordance with an aspect distributing protective cover 10 for use in protecting stethoscope 11. The protective cover dispenser 26 may be constructed of a rigid material, such as but not limited to a plastic that can be mounted to a wall via a wall mount 32. The protective cover dispenser 26 may allow refills of the protective covers with slip in cartridges, or other containers, from either the top or side. For example, the cartridges may contain sets of 10 to 100 or more protective covers. The protective cartridge dispenser 26 may have a cartridge area 34 to receive and hold the cartridges. In an aspect, the protective cover dispenser 26 may include at least 3 rigid sides and an opening in the front. In the front upper portion there may be a flexible material with adhesive 30 on the inner region to allow the protective cover to be suspended with the distal portion open for easy insertion of the bell 18 and tubing 20 portions of the stethoscope 11 into the protective cover 10 (see FIG. 1). The auricular tubes 22 of the stethoscope 11 may be guided by tracks 28 (either rigid or movable) in the anterior/superior regions of the sides of the dispenser 26 for appropriate positioning and easy pinch closure of the protective cover 10 just below the ear piece 24.

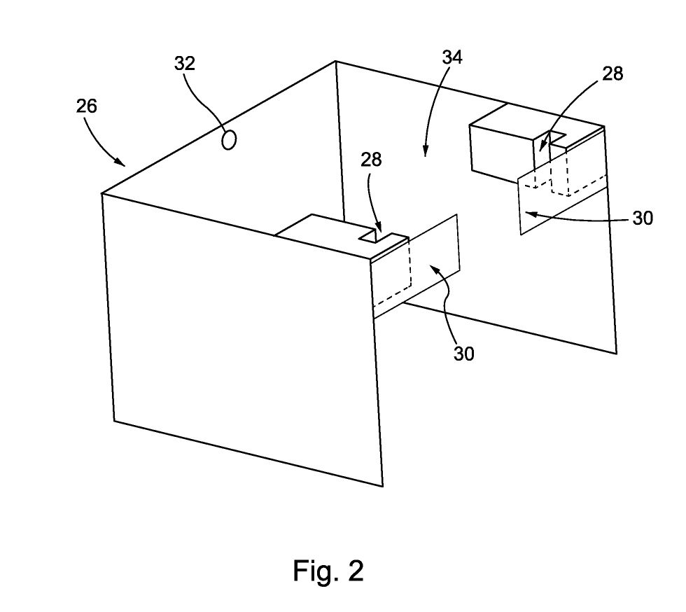

Referring now to FIG. 3, illustrated therein is a top view of a protective cover dispenser 26 with a cartridge 36 positioned in the cartridge area 34 of the protective cover dispenser 26. The flexible material with adhesive 30 on the inner region of the protective cover dispenser 26 allows the protective cover 10 to be suspended with the distal portion open for easy insertion of the bell 18 and tubing 20 portions of the stethoscope 11 into the protective cover 10.

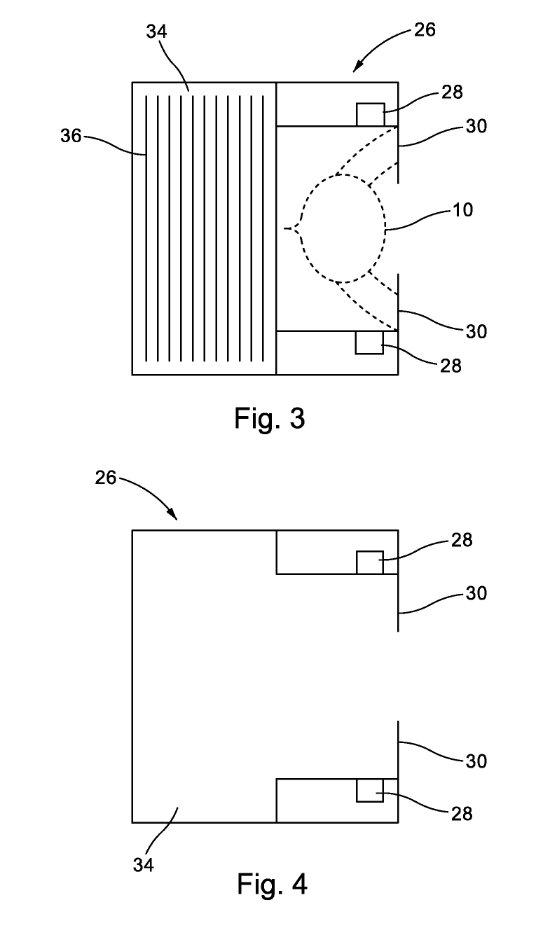

Referring now to FIG. 4, illustrated therein is a top view of a protective cover dispenser 26 (FIG. 2) without a cartridge 36 (see FIG. 3) positioned in the cartridge area 34. Thus, in some aspects, as illustrated in FIGS. 2-4, a protective cover dispenser 26 may include a housing that includes a first side, a second side, and a third side. The first side and the third side may define an opening opposite the second side. In some examples, the protective cover dispenser 26 may include the cartridge area 34 enclosed within the first side, the second side, and the third side. Additionally or alternatively, the protective cover dispenser 26 may further include an inner region configured to suspend a distal portion of a protective cover 10 removed from the cartridge 36, wherein the inner region is between the cartridge area 34 and a first flexible material 30 coupled to the first side and extending into the opening and a second flexible material extending from the third side into the opening.

In yet further examples, the protective cover dispenser 26 may include a first guide track 28 coupled to the first side and extending into the opening and a second guide track 28 coupled to the second side and extending into the opening. The first guide track and the second guide track may be configured to guide the auricular tubes 22 of a stethoscope 11 inserted into the protective cover 10. In some aspects, the first and second guide tracks 28 may be movable or adjustable. In other examples, the first and second guide tracks 28 may be rigid.

While aspects of the present invention have been described in connection with examples thereof, it will be understood by those skilled in the art that variations and modifications of the aspects of the present invention described above may be made without departing from the scope hereof. Other aspects will be apparent to those skilled in the art from a consideration of the specification or from a practice in accordance with aspects of the invention disclosed herein.

1. A protective cover for a stethoscope, comprising:
a distal first tubular portion of a material with an opening configured to receive a bell and tubing of an inserted stethoscope, wherein the distal first tubular portion of the material encases the bell and tubing of the inserted stethoscope; and
a proximal second portion of the material, extended from the distal first tubular portion, configured to receive the auricular tubes of the inserted stethoscope, wherein the proximal second portion includes a one or more adhesive components along the edges of the proximal second portion operable to enclose the proximal second portion of the material around the auricular tubes.

2. The protective cover of claim 1, wherein the proximal second portion of the material is flat.

3. The protective cover of claim 1, further comprising:
a tab coupled to the distal first tubular portion, wherein the tab is configured to remove the protective cover from the stethoscope.

4. The protective cover of claim 1, wherein the material is an impermeable material.

5. The protective cover of claim 1, wherein the material includes an antibacterial powder.

6. A protective cover dispenser, comprising:
a housing including a first side, a second side, and a third side, wherein the first side and the third side define an opening opposite the second side;
a cartridge area enclosed within the first side, the second side, and the third side, wherein the cartridge area is configured to receive a cartridge configured to hold protective covers; and
an inner region configured to suspend a distal portion of a protective cover removed from the cartridge, wherein the inner region is between the cartridge area and a first flexible material coupled to the first side and extending into the opening and a second flexible material extending from the third side into the opening.

7. The protective cover dispenser of claim 6, further comprising:
a first guide track coupled to the first side and extending into the opening; and
a second guide track coupled to the second side and extending into the opening,
wherein the first guide track and the second guide track are configured to guide auricular tubes of a stethoscope inserted into the protective cover.

8. The protective cover dispenser of claim 7, wherein the first guide track and the second guide track are movable.

9. The protective cover dispenser of claim 7, wherein the first guide track and the second guide track are rigid.

10. A method of inserting a stethoscope into a protective cover, comprising:
inserting a bell and tubing of a stethoscope into an opening of a distal tubular portion of a protective cover, wherein the distal tubular portion encases the bell and tubing of the stethoscope with a material;
inserting auricular tubes of the stethoscope into a proximal portion of the protective cover; and
closing one or more adhesive components coupled to the proximal portion of the protective cover, wherein closing the one or more adhesive components encloses the auricular tubes in the material.

11. The method of claim 10, further comprising:
removing the protective cover from the stethoscope by pulling a tab attached to the distal tubular portion.
